(19) United States
(12) Patent Application Publication
(10) Pub. No.: US 2020/0411180 A1
(43) Pub. Date: Dec. 31, 2020

(54) BIOLOGICAL DATA PROCESSING APPARATUS, BIOLOGICAL DATA MEASUREMENT SYSTEM, AND RECORDING MEDIUM

(71) Applicants: Akira KINOSHITA, Tokyo (JP); Shigenori KAWABATA, Tokyo (JP); Masahiro TAKADA, Tokyo (JP); Kazuma GOTO, Ishikawa (JP); Fumikazu HOSHI, Ishikawa (JP); Taishi WATANABE, Tokyo (JP)

(72) Inventors: Akira KINOSHITA, Tokyo (JP); Shigenori KAWABATA, Tokyo (JP); Masahiro TAKADA, Tokyo (JP); Kazuma GOTO, Ishikawa (JP); Fumikazu HOSHI, Ishikawa (JP); Taishi WATANABE, Tokyo (JP)

(73) Assignees: Ricoh Company, Ltd., Tokyo (JP); National University Corporation Tokyo Medical and Dental University, Tokyo (JP)

(21) Appl. No.: 16/911,761

(22) Filed: Jun. 25, 2020

(30) Foreign Application Priority Data
Jun. 26, 2019 (JP) 2019-118971

(51) Int. Cl. G16H 40/63; G16H 30/20

(52) U.S. Cl. CPC G16H 40/63; G16H 30/20

(57) ABSTRACT

A biological data processing apparatus includes an addition averaging processor configured to perform an addition averaging process on measurement data of a magnetic field generated by a living body; an estimator configured to perform an estimation process of estimating an intensity of an action current within the living body based on addition average data acquired by the addition averaging processor; a start instruction acceptor configured to accept a start instruction to start the estimation process by the estimator while measurement of the magnetic field is being performed; and a display configured to display an estimation result obtained by the estimator in response to the start instruction.

### CROSS-REFERENCE TO RELATED APPLICATION

The present application is based on and claims priority under 35 U.S.C. § 119 to Japanese Patent Application No. 2019-118971, filed on Jun. 26, 2019, the contents of which are incorporated herein by reference in their entirety.

### BACKGROUND OF THE INVENTION

### 1. Field of the Invention

The present invention relates to a biological data processing apparatus, a biological data measurement system, and a recording medium.

### 2. Description of the Related Art

In the related art, a conventional apparatus for imaging the intensity of an action current within a living body is known, the action current may be estimated based on the measurement data of a magnetic field generated by a living body, by using a superconducting quantum interference device (SQUID) that is a magnetic sensor (see, for example, Patent Documents 1 to 3).

Further, there is disclosed an apparatus which executes an addition averaging process on the measurement data of the magnetic field, when estimating the intensity of the action current within the living body (see, for example, Patent Documents 4 and 5).
Patent Document 1: Japanese Unexamined Patent Application Publication No. H4-319334
Patent Document 2: Japanese Unexamined Patent Application Publication No. H5-146416
Patent Document 3: Japanese Unexamined Patent Application Publication No. H10-005186
Patent Document 4: Japanese Patent No. 3563624
Patent Document 5: Japanese Patent No. 3230460

### SUMMARY OF THE INVENTION

According to one aspect of the present invention, there is provided a biological data processing apparatus including an addition averaging processor configured to perform an addition averaging process on measurement data of a magnetic field generated by a living body; an estimator configured to perform an estimation process of estimating an intensity of an action current within the living body based on addition average data acquired by the addition averaging processor; a start instruction acceptor configured to accept a start instruction to start the estimation process by the estimator while measurement of the magnetic field is being performed; and a display configured to display an estimation result obtained by the estimator in response to the start instruction.

### BRIEF DESCRIPTION OF THE DRAWINGS

FIG. 1 is a diagram illustrating an overall configuration example of a biological data measurement system according to an embodiment of the present invention;

FIG. 2 is a diagram for explaining a configuration example of a measurement apparatus according to an embodiment of the present invention;

FIG. 3 is a block diagram of an exemplary hardware configuration of a biological data processing apparatus according to an embodiment of the present invention;

FIG. 4 is a block diagram illustrating a functional configuration of a biological data processing apparatus according to a first embodiment of the present invention;

FIG. 5 is a flowchart of a processing example of a biological data processing apparatus according to the first embodiment of the present invention;

FIG. 6 is a diagram illustrating an example of a display screen of measurement data according to the first embodiment of the present invention;

FIG. 7 is a diagram illustrating an example of a display screen during the estimation of an action current according to the first embodiment of the present invention;

FIG. 8 is a diagram illustrating an example of a display screen of an inappropriate result of action current estimation according to the first embodiment of the present invention;

FIG. 9 is a diagram illustrating an example of a display screen of an appropriate result of action current estimation according to the first embodiment of the present invention;

FIG. 10 is a block diagram illustrating a functional configuration of a biological data processing apparatus according to a second embodiment of the present invention;

FIG. 11 is a flowchart of a processing example of a biological data processing apparatus according to the second embodiment of the present invention;

FIG. 12 is a block diagram illustrating a functional configuration of a biological data processing apparatus according to a third embodiment of the present invention;

FIG. 13 is a flowchart of a processing example of a biological data processing apparatus according to the third embodiment of the present invention;

FIG. 14 is a diagram illustrating an example of an accepting screen for specifying an action current estimation position in according to the third embodiment of the present invention; and

FIG. 15 is a diagram illustrating an example of a display screen of an action current estimation result according to the third embodiment of the present invention.

### DETAILED DESCRIPTION OF THE PREFERRED EMBODIMENTS

In apparatuses of the related art, there have been cases where the intensity of the action current within a living body cannot be properly estimated.

A problem to be addressed by an embodiment of the present invention is to properly estimate the intensity of the action current within a living body.

Hereinafter, an embodiment for carrying out the present invention will be described with reference to the drawings. In each drawing, the same parts of the same elements are denoted by the same reference numerals, and overlapping descriptions may be omitted.

In an embodiment, when the magnetic field generated by a living body is being measured, an instruction to start a process to estimate the intensity of the action current within the living body is accepted from a user. Then, based on the addition average data of the measurement data of the magnetic field acquired in response to the start instruction, the intensity of the action current of the living body is estimated, and the estimation result is displayed. The user who has viewed the estimation result determines whether the estimation result is appropriate. If the estimation result is not appropriate, the process of estimating the intensity of the action current is executed again in response to the user's start instruction. If the estimation result is appropriate, the measurement of the biological data is ended.

The action current within a living body is a weak current flowing according to a potential difference caused when an action potential generated when a cell or tissue in a living body receives a stimulus and the stimulated portion is caused to have a negative potential relative to the other portions of the body.

Hereinafter, an embodiment will be described as an example of a biological data measurement system including a measurement apparatus for measuring a magnetic field of a living body and a biological data processing apparatus for processing measurement data obtained by the measurement apparatus. In an embodiment, an example of estimating the intensity of a current flowing through a nerve in the spinal cord in a living body as a result of applying electrical stimulation to the living body, will be described.

The “user” described above and below is a user who uses a biological data measurement system, and more specifically, a doctor who performs medical examination or diagnosis using the biological data measurement system.

### <Overall Configuration of Biological Data Measurement System According to an Embodiment>

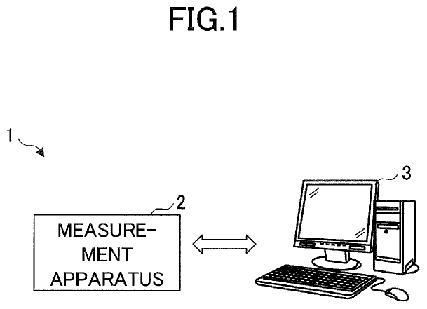

First, the entire configuration of a biological data measurement system 1 according to an embodiment will be described with reference to FIG. 1.

FIG. 1 is a diagram illustrating an example of the overall configuration of the biological data measurement system 1. As illustrated in FIG. 1, the biological data measurement system 1 includes a measurement apparatus 2 and a biological data processing apparatus 3, which are communicably connected to each other in a wired or wireless manner.

The measurement apparatus 2 measures the magnetic field generated by a living body and outputs the measurement data to the biological data processing apparatus 3.

The biological data processing apparatus 3 estimates the intensity of the action current within a living body based on the measurement data input from the measurement apparatus 2, displays the estimation result on a display unit, and stores the estimation result in a storage unit.

### <Example Configuration of the Measurement Apparatus 2 According to an Embodiment>

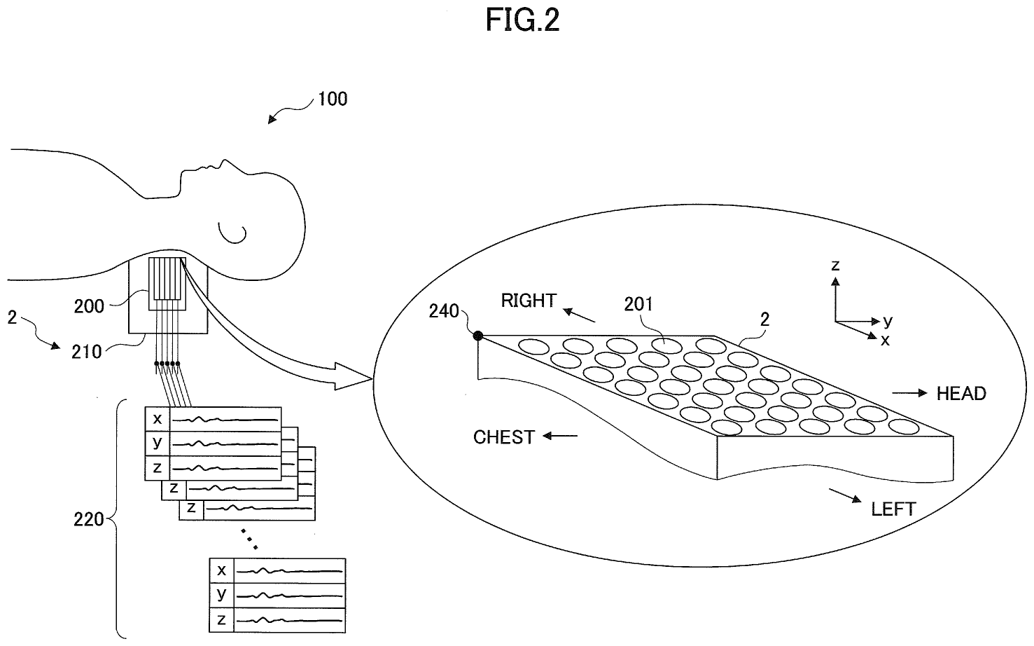

Next, the configuration of the measurement apparatus 2 will be described with reference to FIG. 2.

FIG. 2 is a diagram for explaining an example of a configuration of the measurement apparatus 2. As illustrated in FIG. 2, the measurement apparatus 2 includes a magnetic sensor array 200 and a Dewar 210 that houses the magnetic sensor array 200.

The magnetic sensor array 200 is a bio-sensor with a plurality of magnetic sensors 201 arranged in an array and positioned behind the neck of a subject 100. Here, the subject 100 is an example of a “living body”.

Each of the plurality of magnetic sensors 201 measures the magnetic field of a living body in each direction of the x axis, the y axis, and the z axis illustrated by arrows in FIG. 2 and outputs measurement data. In the example of FIG. 2, the magnetic sensor array 200 includes 7×5 magnetic sensors, and the measurement data obtained by each of the plurality of magnetic sensors 201 is output to the biological data processing apparatus 3. The position where the magnetic sensor array 200 is set with respect to the subject 100 is adjusted in advance using a marker coil or the like.

The interior of the Dewar 210 is filled with liquid helium and is cooled to allow the magnetic sensor array 200 to operate at extremely low temperatures.

In an embodiment, the position of a point 240 on the magnetic sensor array 200 is the origin of the x axis, the y axis, and the z axis. By using the position of the point 240 on the magnetic sensor array 200 as the origin of the x axis, the y axis, and the z axis, the relative positional relationships between the plurality of magnetic sensors 201 in the magnetic sensor array 200 can all be represented by the x coordinates, y coordinates, and z coordinates.

Further, as the method of measuring a magnetic field by the measurement apparatus 2, a publicly known technique described in Japanese Unexamined Patent Application Publication No. 2018-089104 may be applied. Therefore, a more detailed description will be omitted here.

Further, FIG. 2 illustrates an example where the magnetic sensor array 200 is placed behind the neck of the subject 100, but in the following example, the magnetic sensor array 200 is placed behind the waist of the subject 100 and measures magnetic field data within the living body near the waist to estimate the intensity of the current flowing through the nerves in the spinal cord.

### <Hardware Configuration Example of the Biological Data Processing Apparatus 3 According to an Embodiment>

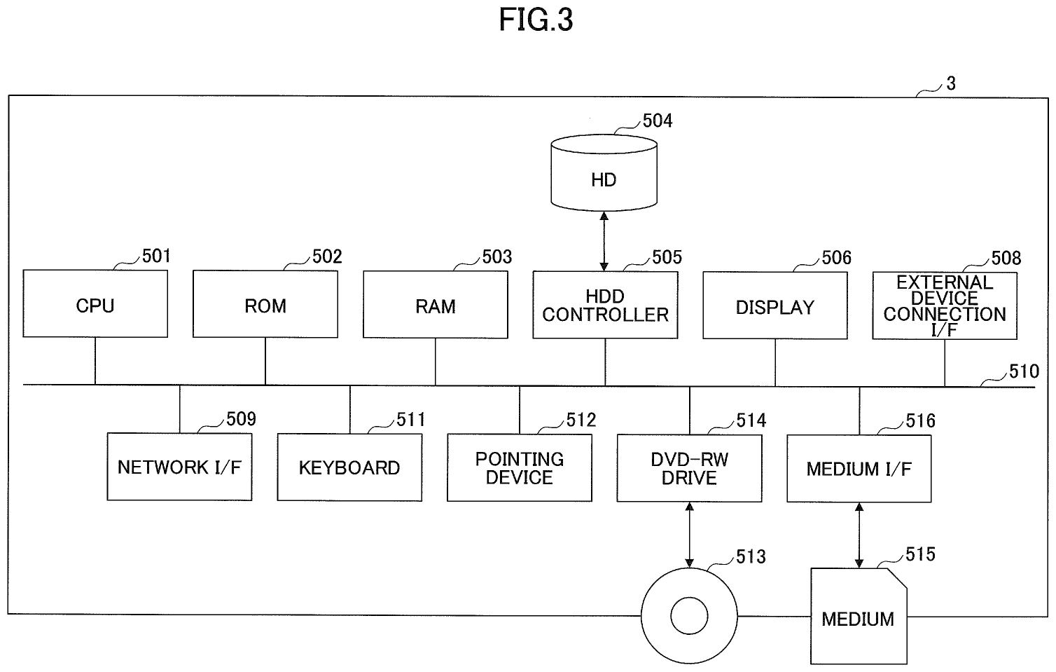

Next, the hardware configuration of the biological data processing apparatus 3 constructed by a computer will be described. FIG. 3 is a block diagram illustrating an example of a hardware configuration of the biological data processing apparatus 3.

As illustrated in FIG. 3, the biological data processing apparatus 3 is constructed by a computer and includes a central processing unit (CPU) 501, a read-only memory (ROM) 502, a random access memory (RAM) 503, a hard disk (HD) 504, a hard disk drive (HDD) controller 505, a display 506, an external device connection I/F 508, and a network I/F 509. Further, the biological data processing apparatus 3 includes a data bus 510, a keyboard 511, a pointing device 512, a Digital Versatile Disk Rewritable (DVD-RW) drive 514, and a medium I/F 516.

Among these, the CPU 501 controls the operation of the entire biological data processing apparatus 3. The ROM 502 stores a program used to drive the CPU 501, such as an IPL (Initial Program Loader).

The RAM 503 is used as a work area of the CPU 501. The HD 504 stores various kinds of data such as programs. The HDD controller 505 controls the reading or writing of various kinds of data to the HD 504 in accordance with the control of the CPU 501.

The display 506 displays various kinds of information such as cursors, menus, windows, characters, or images. The external device connection I/F 508 is an interface for connecting various external devices. In this case, the external device may be, for example, a Universal Serial Bus (USB) memory or a printer.

The network I/F 509 is an interface for performing data communication using a network. The data bus 510 is an address bus, data bus, or the like for electrically connecting components such as the CPU 501 illustrated in FIG. 2.

The keyboard 511 is a type of input unit including a plurality of keys for input of characters, numbers, various indications, and the like. The pointing device 512 is a type of input means for selecting and executing various instructions, selecting a processing target, moving a cursor, and the like. The DVD-RW drive 514 controls the reading or writing of various kinds of data to a DVD-RW 513 as an example of a removable recording medium. The recording medium is not limited to a DVD-RW, but may be a DVD recordable (DVD-R), etc. The medium I/F 516 controls the reading or writing (storage) of data to a recording medium 515, such as a flash memory.

### <Functional Configuration of the Biological Data Processing Apparatus 3 According to a First Embodiment>

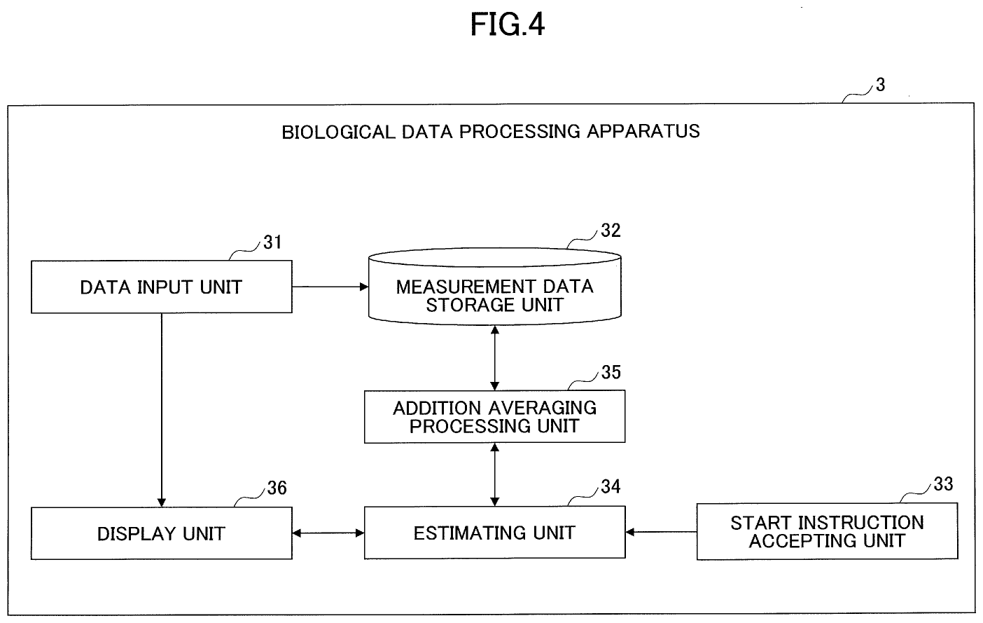

Next, a functional configuration of the biological data processing apparatus 3 according to the first embodiment will be described with reference to FIG. 4. FIG. 4 is a block diagram illustrating an example of a functional configuration of a biological data processing apparatus 3.

As illustrated in FIG. 4, the biological data processing apparatus 3 includes a data input unit 31, a measurement data storage unit 32, a start instruction accepting unit 33, an estimating unit 34, an addition averaging processing unit 35, and a display unit 36.

Among these, the functions of the data input unit 31 are implemented by the external device connection I/F 508 of FIG. 3, and the functions of the measurement data storage unit 32 are implemented by the HD 504 or the like. The function of the start instruction accepting unit 33 is implemented by the CPU 501 executing a predetermined program and the display 506 or the like, and the functions of the estimating unit 34 and the addition averaging processing unit 35 are implemented by the CPU 501 executing a predetermined program or the like. Further, the function of the display unit 36 is implemented by the display 506 or the like.

Among the above-described units, the data input unit 31 inputs the measurement data of the magnetic field acquired by the measurement apparatus 2 at predetermined sampling cycles from the time when the magnetic field measurement starts, and outputs the input measurement data to the measurement data storage unit 32 and the display unit 36. The measurement data storage unit 32 sequentially stores the measurement data input via the data input unit 31.

The start instruction accepting unit 33 accepts an instruction to start the estimation process by the estimating unit 34 from a user and outputs the time at which the start instruction is accepted, to the estimating unit 34, to instruct the estimating unit 34 to start the estimation process. The start instruction accepting unit 33 may accept the start instruction by accepting an instruction output by the CPU 501 at predetermined intervals, in addition to or instead of an instruction from the user.

When the instruction to start a process is accepted from the start instruction accepting unit 33, the estimating unit 34 instructs the addition averaging processing unit 35 to acquire the addition average data and output the acquired addition average data. The estimating unit 34 executes a process of estimating the intensity of the action current within the living body based on the addition average data input from the addition averaging processing unit 35 in response to the instruction and outputs the estimation result to the display unit 36.

The intensity of the action current within the living body is estimated for a rectangularly shaped region or the like within the living body that is at a predetermined distance from the sensing surface of the magnetic sensor array 200. This region is partitioned in a mesh-like manner (a grid) at user-specified intervals, and the intensity of the action current at each opening of the mesh (each open space in the grid) is estimated.

The publicly known technique described in Japanese Unexamined Patent Application Publication No. 2018-089104 can be applied to the above-described process of estimating intensity of the action current, and, therefore, further details will be omitted here. The “process of estimating intensity of the action current” in the embodiment is included in the “reconstruction process of the current source” described in Japanese Unexamined Patent Application Publication No. 2018-089104.

The estimating unit 34 may perform a baseline correction process, a moving average process, a frequency filter process such as a high-pass filter or a low-pass filter, a noise removal process, or the like as a preprocess for estimating the intensity of the action current.

In response to an instruction by the estimating unit 34, the addition averaging processing unit 35 reads the measurement data from the measurement data storage unit 32 obtained during a period from the time when the magnetic field measurement starts to the time when the start instruction accepting unit 33 accepts an instruction to start a process to be performed by the estimating unit 34. Then, the addition average data with respect to the relevant period acquired by calculation using the read-out measurement data, is output to the estimating unit 34. However, the start time and the end time of reading the measurement data may not be the time when the magnetic field measurement is started to the time when the instruction to start the process to be performed by the estimating unit 34 is accepted. For example, the start time and the end time of reading the measurement data may be instructed by the estimating unit 34.

Here, in the related art, the intensity of action current in the spinal cord is often estimated using addition average data acquired based on 4000 to 8000 pieces of measurement data of the magnetic field. On the other hand, according to the embodiment, when 3000 pieces of measurement data are acquired, which is a smaller amount of data compared to the related art, or when 9000 pieces of measurement data are acquired, which is a larger amount of data compared to the related art, the estimating unit 34 can be instructed to start processing. In other words, the number of pieces of the measurement data, which corresponds to the timing when the start instruction from the user is accepted, can be acquired, and the addition average data can be acquired based on the acquired measurement data.

The display unit 36 displays the estimation result input from the estimating unit 34 on the display 506 of FIG. 3. The user may view the estimation result displayed on the display 506 to determine whether the estimation result is appropriate.

Here, a case in which the estimation result is appropriate means a case in which the action current is estimated based on the measurement data of the magnetic field in which noise is sufficiently reduced, because the number of pieces of measurement data used for the processing by the addition averaging processing unit 35 is sufficient. On the other hand, a case in which the estimation result is not appropriate is a case in which the action current is estimated based on the measurement data of the magnetic field including a lot of noise, because the number of pieces of measurement data used for the processing by the addition averaging processing unit 35 is insufficient.

If it is determined that the estimation result is appropriate by the user, the estimation result obtained by the estimating unit 34 is stored in a storage device or output to an external device. On the other hand, if it is determined that the estimation result is not appropriate by the user, the start instruction accepting unit 33 again accepts an instruction to start the estimation process to be performed by the estimating unit 34 from the user, and the estimating unit 34 again executes the estimation process in response to the accepted start instruction.

Here, after the start instruction accepting unit 33 accepts the instruction to start the estimation process from the user, the estimating unit 34 performs the estimation process, and while the display unit 36 displays the estimation result obtained by the estimating unit 34, the measurement apparatus 2 continuously measures the magnetic field, and the measurement data is continuously stored in the measurement data storage unit 32. Therefore, when the estimation result is not appropriate and the estimation process by the estimating unit 34 is executed again, the addition averaging processing unit 35 can read, from the measurement data storage unit 32, the measurement data obtained during the period from the time when the magnetic field measurement is started until the time when the start instruction accepting unit 33 accepts the start instruction again. Accordingly, in the re-estimation process, the estimating unit 34 can execute the estimation process based on the addition average data acquired based on the measurement data in which the number of pieces of measurement data is further increased.

### <Process by the Biological Data Processing Apparatus 3 According to the First Embodiment>

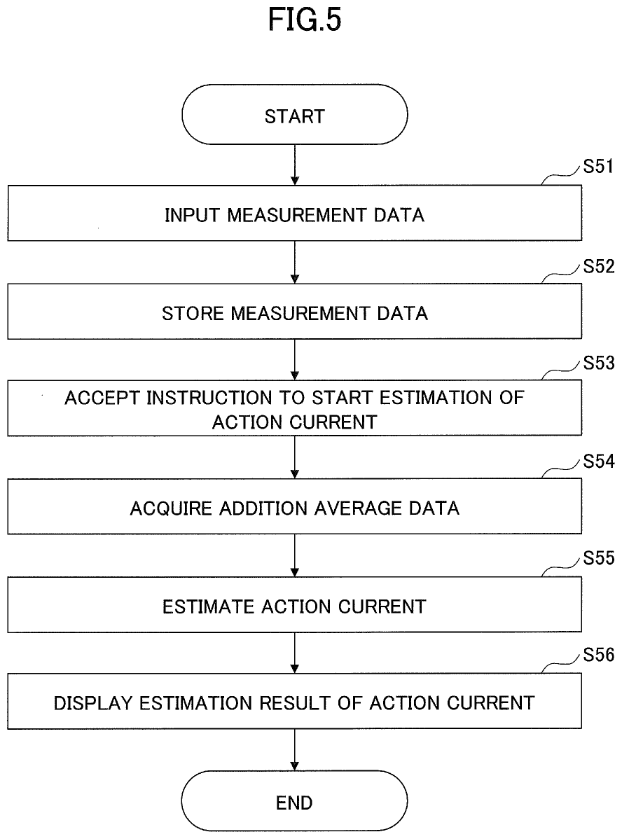

Next, a process by the biological data processing apparatus 3 according to the present embodiment will be described with reference to FIG. 5. FIG. 5 is a flowchart illustrating an example of a process by the biological data processing apparatus 3.

First, in step S51, the data input unit 31 inputs the measurement data of the magnetic field acquired by the measurement apparatus 2 at predetermined sampling cycles from the time when the magnetic field measurement has started, and outputs the measurement data to the measurement data storage unit 32 and the display unit 36.

Subsequently, in step S52, the measurement data storage unit 32 sequentially stores the measurement data input via the data input unit 31.

Subsequently, in step S53, the start instruction accepting unit 33 accepts an instruction to start a process by the estimating unit 34 from the user and outputs the time at which the instruction is accepted to the estimating unit 34, to instruct the estimating unit 34 to start the process. When the instruction to start the process is accepted from the start instruction accepting unit 33, the estimating unit 34 instructs the addition averaging processing unit 35 to acquire the addition average data and output the acquired addition average data.

Subsequently, in step S54, in response to an instruction by the estimating unit 34, the addition averaging processing unit 35 reads, from the measurement data storage unit 32, the measurement data obtained during the period from the time when the magnetic field measurement starts to the time when the start instruction accepting unit 33 accepts the instruction to start an estimation process. Then, the addition average data with respect to the relevant period acquired by calculation using the measurement data that has been read, is output to the estimating unit 34.

Subsequently, in step S55, the estimating unit 34 executes the process of estimating the intensity of the action current within the living body based on the addition average data input from the addition averaging processing unit 35, and outputs the estimation result to the display unit 36.

Subsequently, in step S56, the display unit 36 displays the estimation result input from the estimating unit 34 on the display 506 of FIG. 3.

Thereafter, if the user views the estimation result displayed on the display 506 and determines that the estimation result is appropriate, the measurement by the biological data measurement system 1 ends. On the other hand, if it is determined that the estimation result is not appropriate, the process returns to step S53 and step S53 and beyond are performed again.

In this manner, the biological data processing apparatus 3 can estimate the intensity of the action current within the living body based on the measurement data of the magnetic field, and display the estimation result.

As described above, after the start instruction accepting unit 33 accepts the instruction to start the estimation process from the user in step S53, even while the processes of steps S54 to S56 are executed, the processes of steps S51 and S52 are continued. Therefore, if the estimation result is not appropriate and the processes in step S53 and beyond are executed again, the addition averaging processing unit 35 can read, from the measurement data storage unit 32, the measurement data obtained during the period from the time when the magnetic field measurement starts to the time when the start instruction accepting unit 33 accepts the instruction to start the estimation process again. Accordingly, in the estimation process executed again, the estimating unit 34 can execute the estimation process based on the addition average data acquired based on the measurement data in which the number of pieces of measurement data is further increased.

### <Example of Display Screen According to the Embodiment>

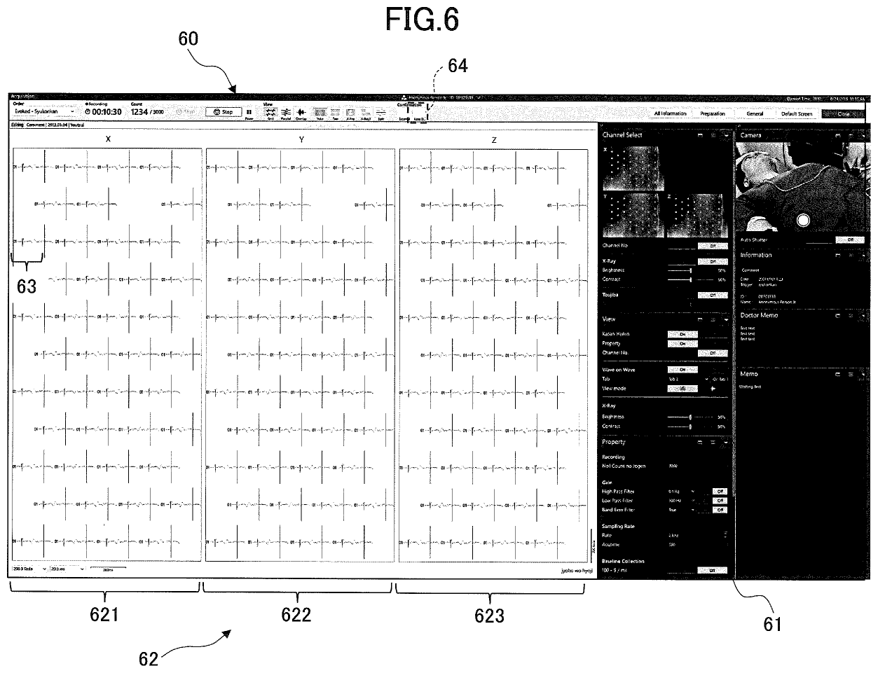

Next, a display screen displayed by the display unit 36 in the biological data measurement system 1 will be described with reference to FIGS. 6 to 9.

FIG. 6 is a diagram illustrating an example of a display screen including measurement data and an operation screen. A measurement display screen 60 includes an operation screen 61 and a measurement data screen 62.

Among these, the operation screen 61 is a screen that is operated by the user to instruct to start the measurement, end the measurement, change the display method of the measurement data, or the like.

The measurement data screen 62 is a screen for displaying the measurement data obtained by the measurement apparatus 2. The measurement data screen 62 includes an x measurement data screen 621, a y measurement data screen 622, and a z measurement data screen 623.

The x measurement data screen 621 displays the measurement data of the magnetic field in the x-axis direction of FIG. 2. The y measurement data screen 622 displays measurement data of the magnetic field in the y-axis direction of FIG. 2, and the z measurement data screen 623 displays measurement data of the magnetic field in the z-axis direction of FIG. 2.

Waveform data 63 displayed on the measurement data screen 62 is for displaying the measurement data obtained by one magnetic sensor included in the magnetic sensor array 200. The horizontal axis of the waveform data 63 indicates time and the vertical axis indicates magnetic field intensity. The waveform data 63 indicates, in real-time, the measurement data obtained by each of a plurality of magnetic sensors included in the magnetic sensor array 200.

The number of pieces the waveform data 63 included in each of the x measurement data screen 621, the y measurement data screen 622, and the z measurement data screen 623 corresponds to the number of the magnetic sensors included in the magnetic sensor array 200.

Here, the addition averaging processing unit 35 (see FIG. 4) acquires the addition average data of the waveform data 63 acquired in time series. Among the data included in the waveform data 63, data corresponding to noise is randomly generated in terms of time, and, therefore, by performing an addition averaging process, the data corresponding to noise is cancelled. On the other hand, among the data included in the waveform data 63, the data representing the magnetic field is accumulated by being added. In this manner, the addition averaging process can amplify data representing the magnetic field compared to data corresponding to noise.

In FIG. 6, a start button 64 represented by a rectangle of a dashed line is a button used by the user to instruct the estimating unit 34 to start the process of estimating the intensity of the action current. When the user presses the start button 64 using the cursor of the pointing device 512 in FIG. 3, the start instruction accepting unit 33 (see FIG. 4) accepts the instruction to start an estimation process to be performed by the estimating unit 34 (see FIG. 4). Here, the start button 64 is an example of a “start instruction acceptor”.

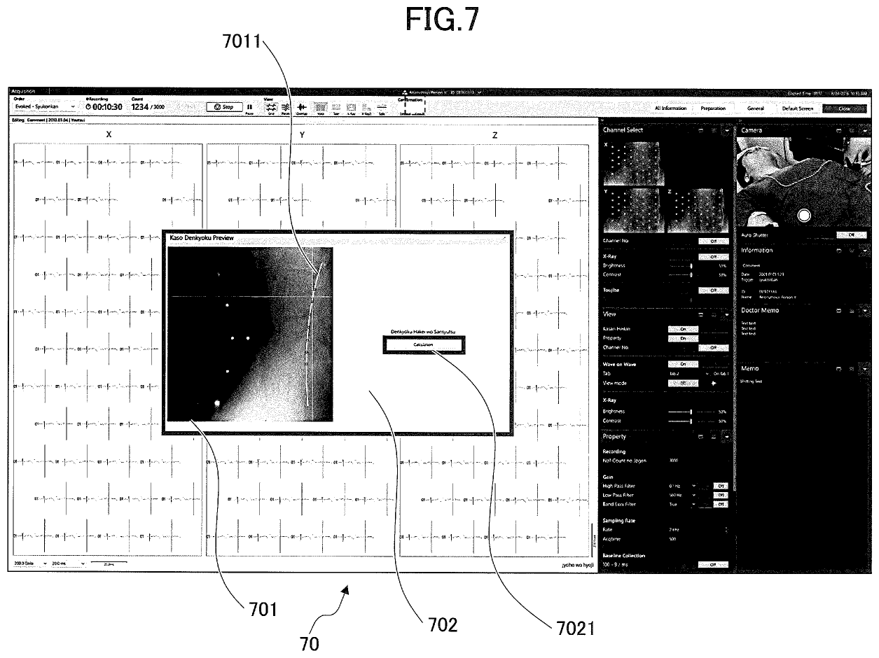

However, after the start button 64 is pressed, a spinal cord position specification screen 70 as illustrated in FIG. 7 may be displayed. The spinal cord position specification screen 70 is a screen used for specifying a different spinal cord position for each subject. As illustrated in FIG. 7, the spinal cord position specification screen 70 includes an X-ray image screen 701 and an estimation start instruction accepting screen 702.

Among these, the X-ray image screen 701 displays an X-ray image from the side of the subject 100 (see FIG. 2). The X-ray image is an image input from an external device via the data input unit 31.

By viewing the X-ray image screen 701 and specifying a point on the screen using the cursor of the pointing device 512 of FIG. 3, the user can specify a position for estimating the intensity of the action current within the living body. The intensity of the action current is estimated within the region including the specified position.

In FIG. 7, a curve 7011 included in the X-ray image screen 701 is a curve automatically rendered to include the point specified on the X-ray image screen 701 by the user, and corresponds to the position of the spinal cord within the subject 100 viewed from the side.

After specifying a point on the curve 7011, the user can press a start button 7021 in the estimation start instruction accepting screen 702 using the cursor of the pointing device 512 of FIG. 3 to cause the start of the process of estimating the intensity of the action current at a position within the subject 100 corresponding to the specified point. Here, the start button 7021 is also an example of a “start instruction accepter” similar to the start button 64.

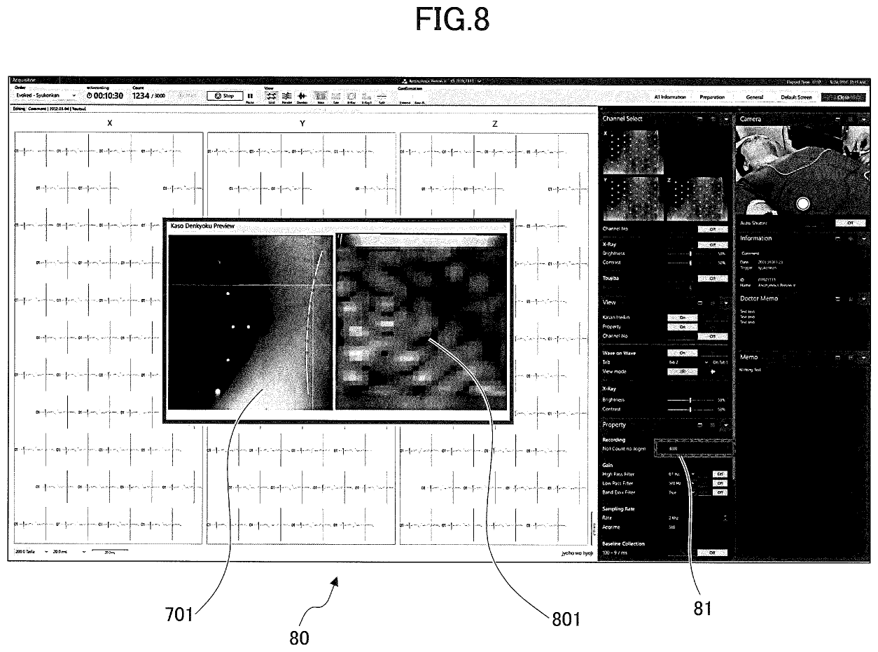

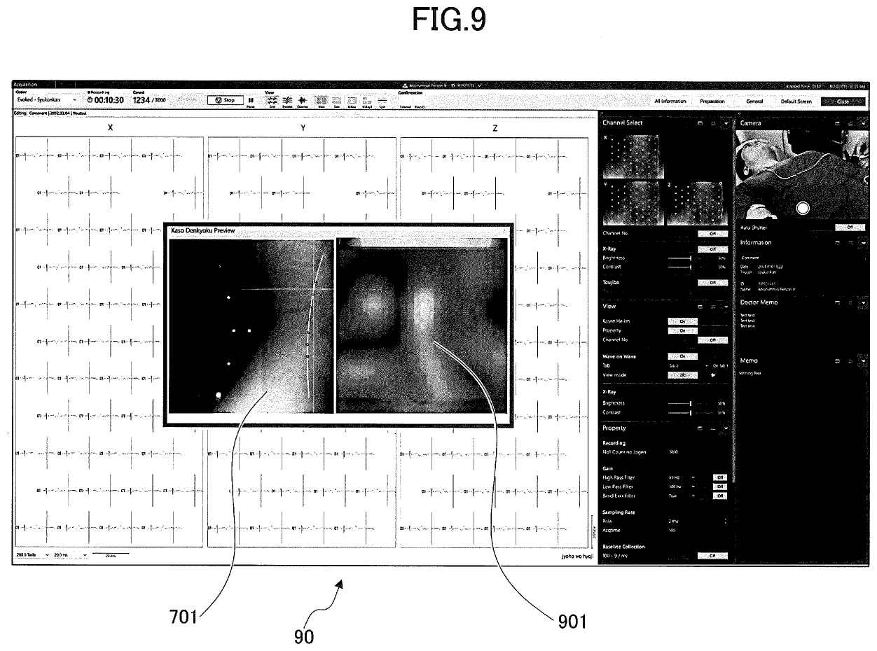

Next, FIGS. 8 and 9 are diagrams illustrating an example of a display screen of an estimation result of an action current. FIG. 8 is a diagram illustrating an example of a display screen of an inappropriate estimation result, and FIG. 9 is a diagram illustrating an example of a display screen of an appropriate estimation result.

In FIG. 8, an estimation result display screen 80 includes the X-ray image screen 701 and a distribution diagram 801 of the intensity of the action current. The distribution diagram 801 is a diagram in which a two-dimensional distribution of the estimated action current intensity is replaced with colors based on data measured by each magnetic sensor included in the magnetic sensor array 200. The biological data measurement system 1 acquires the distribution diagram 801 in time series and displays the distribution diagram 801 in time series to visualize the current flowing through the spinal cord using video images.

In the case of FIG. 8, it can be seen that in the distribution diagram 801, a strong action current is estimated to be outside the human body, where the action current within the living body is not supposed to exist. This indicates that noise has a significant impact on the estimation result of the intensity of the action current. In other words, FIG. 8 illustrates a case of an estimation result that is not appropriate, in which the action current has been estimated based on the measurement data of the magnetic field in which there is a lot of noise because the number of pieces of measurement data used for the process performed by the addition averaging processing unit 35 has been insufficient.

On the other hand, in the case of FIG. 9, it can be seen that in the distribution diagram 901, a strong action current is estimated on the spinal cord of the human body, where the action current is supposed to exist within the living body. This indicates that the impact of noise is reduced in the estimation result of the intensity of the action current. In other words, FIG. 9 illustrates a case of an estimation result that is appropriate, in which the action current is estimated based on the measurement data of the magnetic field in which noise is reduced because the number of pieces of measurement data used for the process performed by the addition averaging processing unit 35 has been sufficient.

The user can view the distribution diagram illustrated in FIGS. 8 and 9 to determine whether the estimation result is appropriate. When it is determined that the estimation result is not appropriate, the user operates the start button 64 (see FIG. 6) to instruct the start of the estimation process again. Instead of accepting the start instruction, the number of pieces of measurement data may be input as a numerical value to an edit box 81 represented by a square of a single dashed line in FIG. 8, and when the number of pieces of measurement data corresponding to the input numerical value is acquired, the estimation process by the estimating unit 34 may be started.

### <Operational Effect of the Biological Data Measurement System 1 According to the First Embodiment>

In the related art, there is known an apparatus that uses a superconducting quantum interference device as a magnetic sensor to image the intensity of an action current within a living body estimated based on measurement data of a magnetic field generated from a living body.

The magnetic field generated by a living body is small, and, therefore, in order to appropriately estimate the action current of a living body, it is desirable to execute an estimation process based on the measurement data in which noise is sufficiently reduced. In contrast, there is disclosed a technique for reducing the impact of noise included in the measurement data by performing an addition averaging process on the measurement data of the magnetic field.

Further, there is disclosed a method of determining whether the number of times of obtaining an addition average is sufficient, by obtaining the addition average using recorded data and displaying, on a screen, information relating to a biological signal waveform and noise calculated from the addition averaging result, other than merely obtaining addition averages corresponding to a specified number of times.

For example, Japanese Patent No. 6055679 discloses a technique of a biological signal addition averaging processing apparatus, of calculating the noise level based on an average value and a standard deviation from an addition average result, presenting a degree of noise reduction by arranging the noise levels in time series, and using the noise levels obtained so far for obtaining a noise level predicted to occur thereafter by linear approximation or curve approximation and presenting the predicted noise level to a user.

Further, in the biological information measurement apparatus and program described in Japanese Unexamined Patent Application Publication No. 2019-076374, a technique is disclosed in which information on the addition average variation is calculated using the confidence interval, and the standard deviation (SD), and the variance from the addition average waveform, and the calculated information is presented to the user along with the addition average waveform.

However, in the related art, the addition averaging process with respect to the magnetic field data can only be performed after measurement of the magnetic field is performed a predetermined number of times. On the other hand, when estimating the intensity of the action current within a living body, the number of times of obtaining addition averages, required to obtain magnetic field data having sufficient quality, varies from patient to patient, and the number of times of obtaining addition averages required to obtain magnetic field data having sufficient quality is not known until confirming the waveform after obtaining the addition average. In particular, in estimating the intensity of the action current of the spinal cord, the number of times of obtaining addition averages required to obtain magnetic field data having sufficient quality varies greatly from patient to patient, and it often takes more than 20 minutes per measurement. In the related art, the addition averaging process can only be performed after measurement of the magnetic field is performed a predetermined number of times. Therefore, there have been cases where the quality of the magnetic field data is found to be insufficient only when the addition averaging process is performed after the completion of measurement. Further, the quality of the magnetic field data required to estimate the intensity of the action current in the spinal cord is difficult to determine simply by confirming the result after the addition averaging process, and it is often possible to determine the quality only by actually estimating the intensity of the action current in the spinal cord. However, because it takes time to estimate the intensity of the action current after the measurement is completed, in order to reduce the burden on the patient's body, patients are often allowed to leave the room in which the measurement is performed before confirming the estimation result. In this case, it may be necessary to perform the measurement again in order to obtain measurement data of the magnetic field having sufficient quality.

In the present embodiment, when the magnetic field is being measured, an instruction to start the process of estimating the intensity of the action current is accepted from the user. Then, the estimation result of the intensity of the action current based on the addition average data of the measurement data of the magnetic field acquired in response to the start instruction, is displayed. The user who has viewed the estimation result determines whether the estimation result is appropriate, and if the estimation result is not appropriate, in response to another start instruction given by the user, the process of estimating the intensity of the action current is executed again.

The process of estimating the intensity of the action current is started at a desired timing before acquiring a predetermined number of pieces of measurement data, and, therefore, the estimation result can is displayed quickly and it can be quickly determined as to whether an appropriate estimation result has been obtained. As a result, the intensity of the action current can be appropriately estimated.

Further, in the present embodiment, an instruction is given to start the estimation process of the intensity of the action current via the start button 64 provided on the screen displayed by the display unit 36. This improves the user operability.

In the present embodiment, the measurement data of the magnetic field and the estimation result are displayed side by side each other (together) on a screen displayed by the display unit 36. Accordingly, the user can more accurately determine whether the estimation result of the intensity of the action current is appropriate based on both the measurement data of the magnetic field and the estimation result.

### Second Embodiment

Next, a biological data measurement system 1a according to the second embodiment will be described.

In the present embodiment, before accepting an instruction to start the process of estimating the intensity of the action current, the addition average data of the measurement data of the magnetic field is acquired at each predetermined time interval Δt, with respect to the measurement data obtained during a period from the time at which the measurement of the magnetic field starts to a time t₀+n·Δt (where n is an integer). Then, the intensity of the action current is estimated based on the acquired addition average data, and, therefore, the processing time for acquiring the addition average data is reduced.

### <Functional Configuration of a Biological Data Processing Apparatus 3a According to the Second Embodiment>

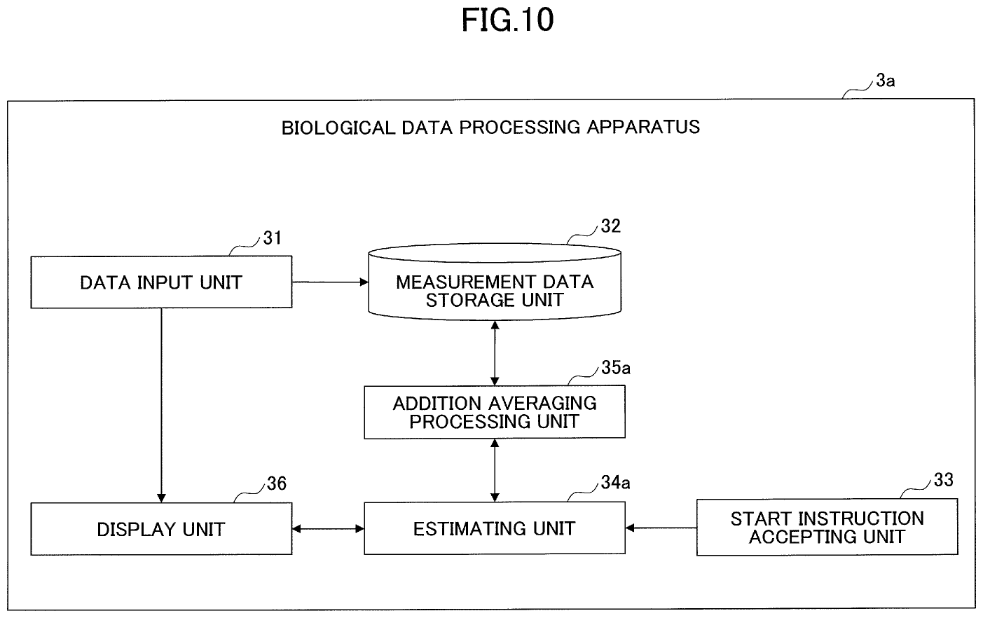

The functional configuration of the biological data processing apparatus 3a according to the present embodiment will be described with reference to FIG. 10. FIG. 10 is a block diagram illustrating an example of a functional configuration of the biological data processing apparatus 3a. As illustrated in FIG. 10, the biological data processing apparatus 3a includes an estimating unit 34a and an addition averaging processing unit 35a. The functions of the estimating unit 34a and the addition averaging processing unit 35a are implemented by, for example, the CPU 501 of FIG. 3 executing a predetermined program.

When an instruction to start a process is accepted from the start instruction accepting unit 33, the estimating unit 34a instructs the addition averaging processing unit 35a to output the addition average data. The estimating unit 34a executes the process of estimating the intensity of the action current within a living body based on the addition average data input from the addition averaging processing unit 35a and outputs the estimation result to the display unit 36.

Before the start instruction accepting unit 33 accepts the start instruction for the process to be performed by the estimating unit 34a, the addition averaging processing unit 35a reads, from the measurement data storage unit 32 at each predetermined time interval Δt, the measurement data obtained during a period from the time t₀ at which the measurement of the magnetic field by the measurement apparatus 2 starts to a time t₀+n·Δt. Then, the addition averaging processing unit 35a acquires the addition average data for the relevant period by calculation based on the measurement data that has been read, and outputs the addition average data to the estimating unit 34a in response to an instruction from the estimating unit 34a. More specifically, the addition averaging processing unit 35a outputs, to the estimating unit 34a in response to the instruction from the estimating unit 34a, the addition average data, which had already been acquired at a time earlier than the time at which the instruction has been given by the estimating unit 34a and nearest to the corresponding time.

### <Process by the Biological Data Processing Apparatus 3a According to the Second Embodiment>

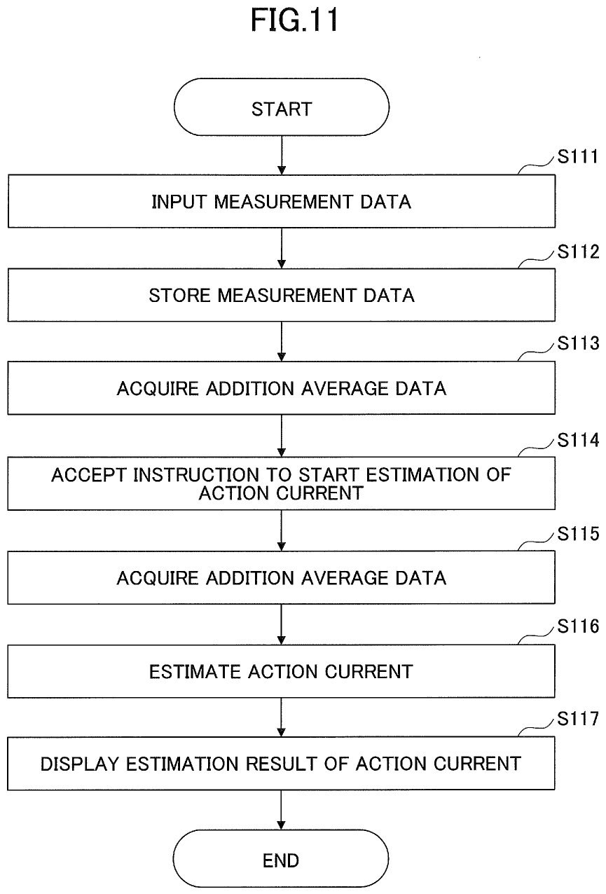

Next, a process by the biological data processing apparatus 3a will be described with reference to FIG. 11. FIG. 11 is a diagram illustrating an example of a process performed by the biological data processing apparatus 3a. The processes of steps S111 and S112 are similar to the processes of steps S51 and S52 in FIG. 5, and the processes of steps S116 and S117 are similar to the processes of steps S55 and S56 in FIG. 5, and, therefore, the overlapping descriptions will be omitted here.

In step S113, before the start instruction accepting unit 33 accepts the start instruction for the process to be performed by the estimating unit 34a, the addition averaging processing unit 35a reads, from the measurement data storage unit 32 at each predetermined time interval Δt, the measurement data obtained during a period from the time t₀ at which the measurement of the magnetic field by the measurement apparatus 2 starts to a time t₀+n·Δt. Then, the addition average data for the relevant period is acquired by calculation based on the measurement data that has been read.

Subsequently, in step S114, the start instruction accepting unit 33 accepts an instruction to start a process to be performed by the estimating unit 34a from the user and outputs the time at which the instruction is accepted to the estimating unit 34a to instruct the estimating unit 34a to start the process. When the processing start instruction is accepted from the start instruction accepting unit 33, the estimating unit 34a instructs the addition averaging processing unit 35a to output the addition average data.

Subsequently, in step S115, the addition averaging processing unit 35a outputs, to the estimating unit 34a in response to the instruction from the estimating unit 34a, the addition average data, which had already been acquired at a time earlier than the time at which the instruction has been given by the estimating unit 34a and nearest to the corresponding time.

In this manner, the biological data processing apparatus 3a is capable of estimating the intensity of the action current within a living body based on measurement data of a magnetic field, and displaying the estimation result.

### <Effect of the Biological Data Measurement System 1a According to the Second Embodiment>

It may take a long time to execute the addition averaging process of measurement data of a magnetic field. This becomes more significant as the number of pieces of measurement data increases.

In the present embodiment, before accepting the start instruction for the process of estimating the intensity of the action current, the addition average data of the measurement data of the magnetic field is acquired at each predetermined time interval Δt, with respect to measurement data obtained during a period from the time t₀ at which the measurement of the magnetic field starts to a time t₀+n·Δt. Then, the intensity of the action current is estimated based on the acquired addition average data. When the start instruction accepting unit 33 accepts the start instruction for the estimation process, the addition averaging processing unit 35a is able to output the addition average data that is already acquired, thereby significantly reducing the processing time for acquiring the addition average data.

The effects other than those described above are the same as those described in the first embodiment.

### Third Embodiment

Next, a biological data measurement system 1b according to the third embodiment will be described.

In the present embodiment, a specification of a position within a living body, at which the intensity of the action current is to be estimated, is accepted, and the intensity of the action current at the relevant position is estimated and displayed.

### <Functional Configuration of a Biological Data Processing Apparatus 3b According to the Third Embodiment>

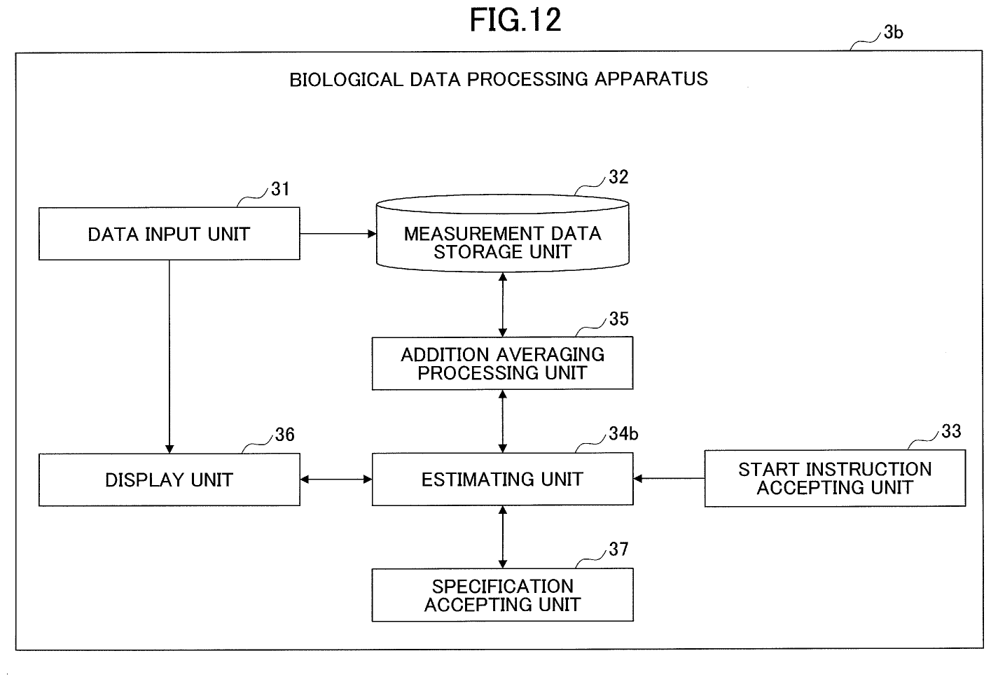

The functional configuration of the biological data processing apparatus 3b according to the third embodiment will be described with reference to FIG. 12. FIG. 12 is a block diagram illustrating an example of a functional configuration of the biological data processing apparatus 3b. As illustrated in FIG. 12, the biological data processing apparatus 3b includes a specification accepting unit 37 and an estimating unit 34b.

Among these, the function of the specification accepting unit 37 is implemented by the CPU 501 executing a predetermined program and the display 506 or the like, and the function of the estimating unit 34b is implemented by the CPU 501 executing a predetermined program or the like.

Among the above-described units, the specification accepting unit 37 accepts, from a user, a specification of a position within the living body at which the intensity of the action current is to be estimated by the estimating unit 34b, and outputs information representing the specified position to the estimating unit 34b. The information representing such a position is coordinate data in the x axis direction and the y axis direction in FIG. 2.

The estimating unit 34b estimates the intensity of the action current at the specified position and outputs the estimation result to the display unit 36.

### <Process by the Biological Data Processing Apparatus 3b According to the Third Embodiment>

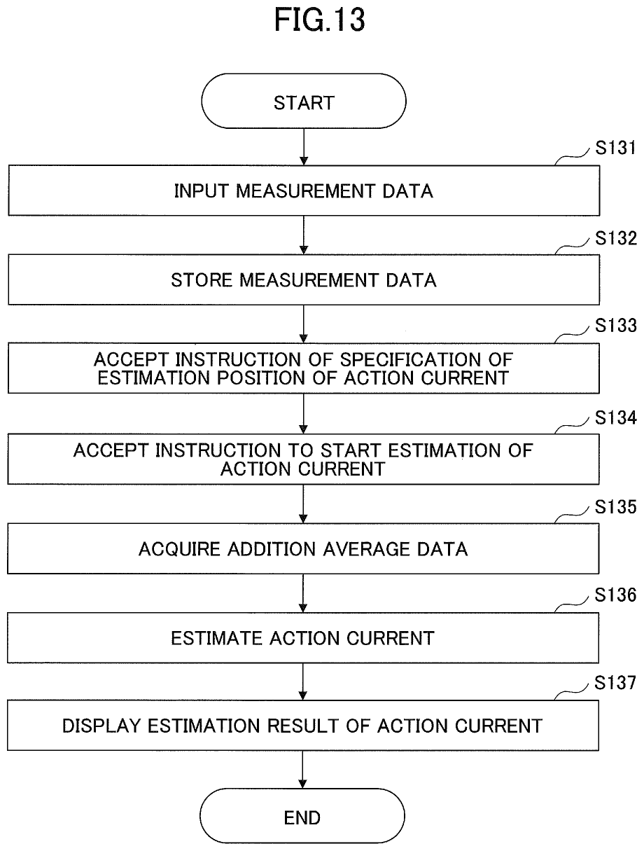

The process by the biological data processing apparatus 3b according to the third embodiment will be described with reference to FIG. 13. FIG. 13 is a flowchart illustrating an example of process by the biological data processing apparatus 3b. The processes of steps S131 and S132 are the same as the processes of steps S51 and S52 in FIG. 5, and, therefore, the overlapping descriptions will be omitted here.

In step S133, the specification accepting unit 37 accepts, from the user, a specification of a position within the living body, at which the intensity of the action current is to be estimated by the estimating unit 34b, and outputs information representing the specified position to the estimating unit 34b.

Subsequently, in step S134, the start instruction accepting unit 33 accepts an instruction to start a process to be performed by the estimating unit 34b from the user, outputs the time at which the instruction is accepted to the estimating unit 34b, and instructs the estimating unit 34b to start the process. When the process start instruction is accepted from the start instruction accepting unit 33, the estimating unit 34b instructs the addition averaging processing unit 35 to acquire the addition average data and output the acquired addition average data.

Subsequently, in step S135, in response to the instruction by the estimating unit 34b, the addition averaging processing unit 35 reads the measurement data from the measurement data storage unit 32 obtained during the period from the time when the magnetic field measurement starts to the time when the start instruction accepting unit 33 accepts the instruction to start the process to be performed by the estimating unit 34b. Then, the addition averaging processing unit 35 outputs, to the estimating unit 34b, the addition average data in the relevant period acquired by calculation based on the measurement data that has been read.

Subsequently, in step S136, the estimating unit 34b executes a process for estimating the intensity of the action current at the position specified in step S133 based on the addition average data acquired based on the measurement data at the position specified in step S133, input from the addition averaging processing unit 35, and outputs the estimation result to the display unit 36.

Subsequently, in step S137, the display unit 36 displays the estimation result of the action current of the living body input from the estimating unit 34b, on the display 506 of FIG. 3.

In this manner, the biological data processing apparatus 3b is able to estimate the intensity of the action current within a living body based on the measurement data of the magnetic field and display the estimation result.

### <Example of Display Screen According to the Third Embodiment>

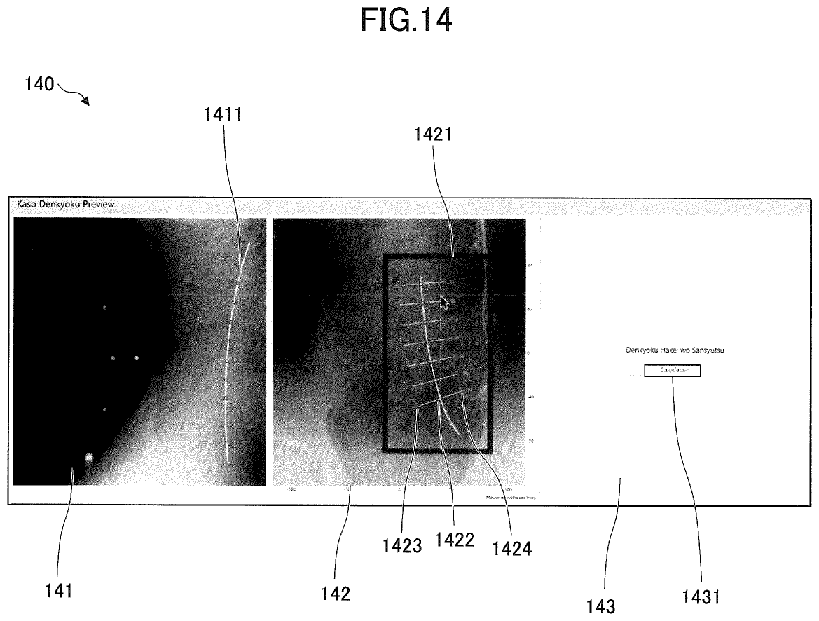

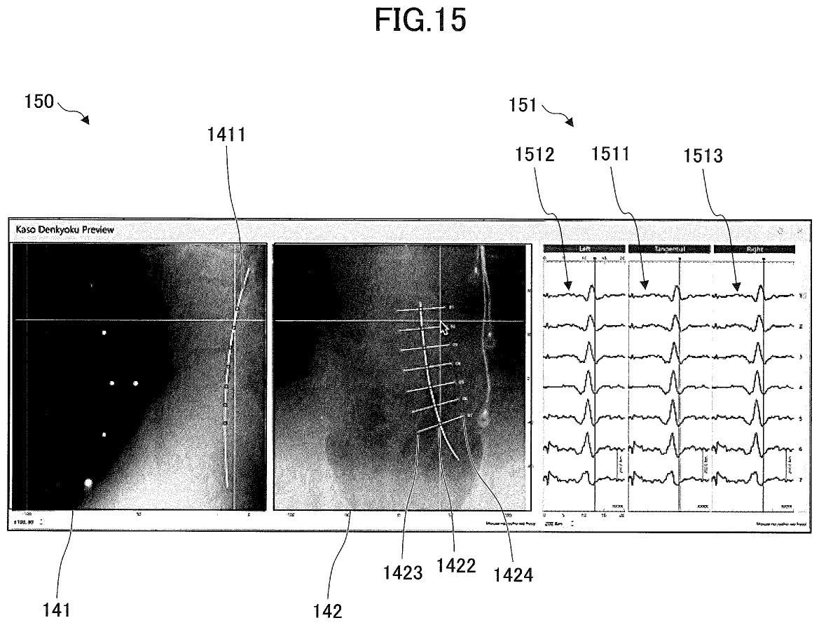

Next, a display screen displayed by the display unit 36 in the biological data measurement system 1b will be described with reference to FIGS. 14 and 15.

FIG. 14 is a diagram illustrating an example of a display screen for accepting the specification of an estimation position of an action current. As illustrated in FIG. 14, an estimation position specification screen 140 includes a side X-ray image screen 141, a front X-ray image screen 142, and an estimation start instruction accepting screen 143. Each of the side X-ray image screen 141 and the front X-ray image screen 142 is an example of a specification acceptor.

The side X-ray image screen 141 displays an X-ray image capturing the side of the subject 100 (see FIG. 2). The front X-ray image screen 142 displays an X-ray image capturing the front of the subject 100. Each of these X-ray images is an image input from an external device via the data input unit 31.

The user can view the side X-ray image screen 141 and/or the front X-ray image screen 142 and specify a position at which the intensity of the action current is to be estimated, by specifying a point on the screen using the cursor of the pointing device 512 of FIG. 3.

In FIG. 14, a curve 1411 included in the side X-ray image screen 141 is a curve automatically rendered to connect the points specified on the side X-ray image screen 141 by the user, similar to the case as described in FIG. 7, and corresponds to the position of the spinal cord within the living body viewed from the side.

In the front X-ray image screen 142, curves 1422 to 1424 are displayed in a region 1421 that is surrounded by a dashed line rectangle. The curve 1422 is a curve rendered automatically to include points specified by the user on the front X-ray image screen 142. Also, each of the curves 1423 and 1424 is an automatically rendered curve connecting a point on the curve 1422 and a point distant from the curve 1422 in a normal direction by a predetermined distance.

Preferably, the position for estimating the intensity of the action current is specified such that the center of the vertebra and the center of the intervertebral disc on the spinal cord are included in the position within the living body corresponding to the points on these curves 1422 to 1424.

After specifying points on the curves 1422 to 1424, the user can press a start button 1431 on the estimation start instruction accepting screen 143 using the cursor of the pointing device 512 illustrated in FIG. 3 to provide an instruction to start the estimation process of the intensity of the action current at a position within the living body corresponding to the points specified on the screen. Here, the start button 1431 is an example of a “start instruction acceptor”.

Next, FIG. 15 is a diagram illustrating an example of a display screen of an action current estimation result. As illustrated in FIG. 15, an estimation result display screen 150 includes the side X-ray image screen 141, the front X-ray image screen 142, and an estimation result screen 151.

The side X-ray image screen 141 and the front X-ray image screen 142 are the same as those illustrated in FIG. 14, and, therefore, the descriptions thereof will be omitted here.

The estimation result screen 151 includes seven pieces of waveform data 1511 representing the variation over time of the intensity of the action current at a position within the living body corresponding to the seven points on the curve 1422. The estimation result screen 151 also includes seven pieces of waveform data 1512 representing the variation over time of the intensity of the action current at a position within the living body corresponding to the seven points on the curve 1423 and seven pieces of waveform data 1513 representing the variation over time of the intensity of the action current at a position within the living body corresponding to the seven points on the curve 1424. The vertical axis of the waveform data 1511 to 1513 indicates the intensity of the action current.

Large peaks occur near the center of each waveform included in the waveform data 1511 to 1513. The data other than the peak correspond to noise, and the noise is sufficiently small as compared to the intensity of the action current at the peak.

### <Effect of the Biological Data Processing Apparatus 3b According to the Third Embodiment>

In the description of the first embodiment, in the distribution diagrams 801 and 901 illustrated in FIGS. 8 and 9, there are many positions at which the intensity of the action current needs to be estimated, and, therefore, it may take time to perform the estimation process. In contrast, in the present embodiment, a specification of a position within a living body, at which the intensity of the action current is to be estimated, is accepted, and the intensity of the action current at the position is estimated and displayed. Therefore, it is sufficient to estimate the intensity of the action current at only the specified position, which reduces the time required for the estimation process. Further, the intensity of the action current at the appropriate position can be estimated by viewing the X-ray image and specifying a point corresponding to the position of the spinal cord.

The effects other than those described above are the same as those described in the first and second embodiments.

According to one embodiment of the present invention, it is possible to properly estimate the intensity of the action current within a living body.

The biological data processing apparatus, the biological data measurement system, and the recording medium are not limited to the specific embodiments described in the detailed description, and variations and modifications may be made without departing from the spirit and scope of the present invention.

These embodiments also encompass programs. For example, a program causes a processor to perform an addition averaging process on the measurement data of a magnetic field generated from a living body, estimates the intensity of the action current within the living body based on the data acquired by the addition averaging process, accepts an instruction to start a process to estimate the intensity of the action current while the magnetic field is being measured, and displays the estimation result obtained by the estimating unit in response to the start instruction. Such a program may have the same effects as those of the biological data processing apparatus described above.

The functions of each of the embodiments described above may be implemented by one or more processing circuits. As used herein, a “processing circuit” includes a processor programmed to execute each function by software such as a processor implemented in an electronic circuit; or devices such as an Application Specific Integrated Circuit (ASIC) a digital signal processor (DSP), a field programmable gate array (FPGA), and a conventional circuit module, designed to execute each function as described above.

What is claimed is:

1. A biological data processing apparatus comprising:
an addition averaging processor configured to perform an addition averaging process on measurement data of a magnetic field generated by a living body;
an estimator configured to perform an estimation process of estimating an intensity of an action current within the living body based on addition average data acquired by the addition averaging processor;
a start instruction acceptor configured to accept a start instruction to start the estimation process by the estimator while measurement of the magnetic field is being performed; and
a display configured to display an estimation result obtained by the estimator in response to the start instruction.

2. The biological data processing apparatus according to claim 1, wherein the addition averaging processor performs the addition averaging process on the measurement data of the magnetic field obtained during a period from a time at which the measurement of the magnetic field starts to a time when the start instruction is accepted.

3. The biological data processing apparatus according to claim 1, wherein before the start instruction is accepted, at each predetermined time interval Δt, the addition averaging processor performs the addition averaging process on the measurement data of the magnetic field obtained during a period from a time t₀ at which the measurement of the magnetic field starts to a time t₀+n·Δt.

4. The biological data processing apparatus according to claim 1, wherein the start instruction acceptor is provided on a screen displayed by the display.

5. The biological data processing apparatus according to claim 1, wherein the display displays the measurement data of the magnetic field and the estimation result obtained by the estimator side by side each other.

6. The biological data processing apparatus according to claim 1, further comprising:
a specification acceptor configured to accept a specification of a position within the living body at which the intensity of the action current is to be estimated, wherein
the estimator estimates the intensity of the action current at the position.

7. The biological data processing apparatus according to claim 6, wherein the estimator estimates the intensity of the action current at the position corresponding to at least one of a point on a curve connecting points on a screen displayed by the display and a point distant from the point on the curve in a normal direction of the curve by a predetermined distance, accepted by the specification acceptor.

8. The biological data processing apparatus according to claim 7, wherein the position includes a center of a vertebra and a center of an intervertebral disc on a spinal cord of the living body.

9. A biological data measurement system comprising:
a measurement apparatus configured to measure a magnetic field generated from a living body;
a biological data processing apparatus configured to process measurement data obtained by the measurement apparatus;
an addition averaging processor configured to perform an addition averaging process on the measurement data of the magnetic field obtained by the measurement apparatus;
an estimator configured to perform an estimation process of estimating an intensity of an action current within the living body based on addition average data acquired by the addition averaging processor;
a start instruction acceptor configured to accept a start instruction to start the estimation process by the estimator while measurement of the magnetic field is being performed by the measurement apparatus; and
a display configured to display an estimation result obtained by the estimator in response to the start instruction.

10. A non-transitory computer-readable recording medium storing a program that causes a computer to execute a process, the process comprising:
performing an addition averaging process on measurement data of a magnetic field generated from a living body;
estimating an intensity of an action current within the living body based on addition average data acquired by the addition averaging process;
accepting a start instruction to start the estimating of the intensity of the action current while measurement of the magnetic field is being performed; and
displaying an estimation result obtained at the estimating in response to the start instruction.
